(19) United States
(12) Patent Application Publication
(10) Pub. No.: US 2010/0298816 A1
(43) Pub. Date: Nov. 25, 2010

(54) MAGNETIC DELIVERY DEVICE

(75) Inventor: Jon Dobson, Staffordshire (GB)

Correspondence Address: SPECKMAN LAW GROUP PLLC, 1201 THIRD AVENUE, SUITE 330, SEATTLE, WA, 98101, US

(73) Assignee: KEELE UNIVERSITY, Staffordshire (GB)

(21) Appl. No.: 12/677,285

(22) PCT Filed: Sep. 10, 2008
(86) PCT No.: PCT/GB2008/003069

§ 371 Date: Jul. 1, 2010

(30) Foreign Application Priority Data
Sep. 10, 2007 (GB) 0717582.1

(51) Int. Cl. A61M 37/00; C12N 13/00; C12M 1/00; A61K 9/22; B23P 11/00

(52) U.S. Cl. 604/891.1; 435/173.5; 435/283.1; 600/12; 29/428

(57) ABSTRACT

A method of delivering a reagent into a cell comprising positioning at least one cell, and at least one magnetically susceptible particle attached to the reagent, in the magnetic field of a Halbach array such that the magnetically susceptible particle is attracted to and contacts the cell is described together with an apparatus for delivery of the reagent.

### FIELD OF THE INVENTION

The present invention relates to the field of biotechnology and, in particular, methods and apparatus for delivering a reagent into a cell. The present invention encompasses an apparatus for use in the transfection of living cells with nucleic acid using magnetically susceptible particles to deliver the nucleic acid. The invention also relates to methods of delivering a reagent into a cell, including methods of transfection, using such apparatus.

### BACKGROUND

The introduction of exogenous reagents into living cells has many potential utilities in both biotechnological and clinical settings. Many techniques for delivering such agents have been developed, each with different advantages and disadvantages. Much work to date has been done in the field of delivering exogenous nucleic acids into cells with a view to transfecting the cells.

The introduction of exogenous nucleic acids into living cells by the process of transfection is a key, and now routine, process in many areas of the biosciences and biotechnology. For small-scale laboratory procedures, this was originally achieved by means of techniques such as calcium phosphate precipitation of naked DNA vectors, but rapidly a variety of improved techniques was developed, including electroporation, complexation with asbestos, polybrene, DEAE, dextran, liposomes, lipopolyamines, polyornithine, polycationic peptides, particle bombardment and direct microinjection (reviewed by Kucherlapati and Skoultchi (1984) Crit. Rev. Biochem. 16:349-79; Keown et al. (1990) Methods Enzymol. 185: 527). The most widely-used non-viral laboratory transfection techniques are probably electroporation and the use of cationic lipid-based formulations, with many commercial products being available.

Where the purpose of transfection is to introduce genetic material that is then transcribed and translated, so-called expression vectors, adapted for gene expression in the appropriate cell type are used. For many purposes, eukaryotic, and often mammalian cells are used, with appropriately designed eukaryotic expression vectors. Typically these are provided with transcription control sequences (promoter and enhancer sequences), which mediate expression. Adaptations also include the provision of selectable markers and autonomous replication sequences which both facilitate the maintenance of said vector in the host cell. Vectors that are maintained autonomously are referred to as episomal vectors and they are useful since they are self-replicating and so persist without the need for integration.

Adaptations which facilitate the expression of vector-encoded genes also include the provision of transcription termination/polyadenylation sequences. This also includes the provision of internal ribosome entry sites (IRES) which function to maximise expression of vector encoded genes arranged in bicistronic or multi-cistronic expression cassettes. For specialised applications, where large volumes of cells need to be transfected with high efficiency, or for clinical gene therapy applications, viral vectors based on modified viruses such as adenoviruses or lentiviruses are often employed. However, for many purposes, viral vectors introduce unnecessary complexity and safety considerations in both their production and use, and have limited cloning capacity.

These techniques and vectors are well-known in the art. There is a significant amount of published literature with respect to expression vector construction and recombinant DNA techniques in general. Please see, Sambrook et al (1989) Molecular Cloning: A Laboratory Manual, Cold Spring Harbour Laboratory, Cold Spring Harbour, N.Y. and references therein; Marston, F (1987) DNA Cloning Techniques: A Practical Approach Vol III IRL Press, Oxford UK; DNA Cloning: F M Ausubel et al, Current Protocols in Molecular Biology, John Wiley & Sons, Inc. (1994). Of increasing importance is the transfection of other nucleic acids, in particular double-stranded iRNA 10 molecules for targeted interference with transcription, DNA or RNA aptamers, and ribozymes (An, C I et al. (2006) RNA 12(5):710-716; Barrandon, C et al., (2008) Biol Cell. 100(2):83-95; Nguyen, T et al. (2008) Curr Opin Mol Ther. 10(2):158-167; Pan W and G A Clawson (2008) Expert Opin Biol Ther. 8(8):1071-1085). In addition, further macromolecules such as polypeptides may be introduced by similar methods, either in combination with nucleic acids, or alone.

However, known non-viral techniques suffer from significant drawbacks such as: (i) low levels of transfection in primary cells and some cell lines (ii) their inability to effectively transfect tissue explants (iii) detrimental effects on cell viability (primarily with electroporation) and (iv) difficulty in translating to in vivo (clinical) applications. There is, therefore, a need for non-viral transfection techniques which can overcome these obstacles.

One of the more recent physical approaches that has been developed for improving the efficiency of transfection both in vitro and in vivo is the use of magnetic nanoparticles (Mah et al. (2000) Molecular Therapy 1(5): S239; Mah et al (2002) Molecular Therapy 6: 106-112; Scherer et al, 2002, Gene Therapy ˜:102; International patent application WO 02/00870). The technique involves coupling DNA or other nucleic acid to biocompatible magnetic nanoparticles which are used as carriers. The gene/particle complex is then targeted to cells via high-gradient, rare earth (usually NdFeB) magnets which are focused over the target site or placed beneath the culture dish. These magnets produce a translational force on the particles due to their high field strength/gradient product, which effectively “pulls” the particles into contact with the cells (Dobson (2006) Gene Therapy 13: 283-287).

Such systems employ an array of small cylindrical or disc magnets, each producing a field that interacts little with its neighbour. The arrays are positioned beneath the receptacle containing the cells to be transfected, often a standard 96- or 24-well plate, or a conventional tissue culture flask. This means that there are certain restrictions on the field gradient and strength that can be produced, and the force to which the magnetic nanoparticles and cells are subjected may vary significantly depending on their position within the well or flask.

The use of pulsed magnetic fields for transfection is disclosed in International patent application U.S. Pat. No. 5,753,477 and the use of oscillating fields generated by moving magnets is taught by International patent application WO 2006/111770.

International patent application WO 2007/018562 discloses the use of a specifically structured, elongated magnetic nanostructure for delivering biomolecules such as DNA into cells by means of an applied magnetic force.

Halbach arrays are arrangements of adjacent individual magnets in a specific sequence of orientations of their poles as shown in FIG. 1, such that there is an additive effect on the magnetic field on one side of the array while the field on the other side is effectively cancelled. The net effect is effectively an array with a one-sided flux (Mallinson, 1973, IEEE Transactions on Magnetics 9: 678; K. Halbach, 1981, Nucl. Inst. and Methods 187. pp. 109-117). The field produced is not only approximately twice the strength of that obtained by a conventional array, but is also highly contained, producing a high field gradient. It is this combination of strong field and high gradient that produces extra force on magnetically susceptible particles exposed to the array.

### SUMMARY OF THE INVENTION

The current invention discloses methods and apparatus allowing the use of a high-gradient magnetic field to provide significantly improved performance for the magnetic delivery of reagents to cells and other applications. The device comprises a Halbach array (for example, FIG. 1), which may be an array of permanent magnets configured to accelerate magnetically susceptible particles coupled to reagents, such as DNA, RNA or other nucleic acid onto cells. In addition to single- and double-stranded DNA or RNA (including iRNA), the device and method may equally be used to insert any of a variety of other molecules and moieties into cells by coupling them to suitable magnetically susceptible particles. Such molecules include non-coding nucleic acids such as ribozymes and nucleic acid based aptamers, peptides and proteins (including those capable of binding specific intracellular targets), modified peptides and proteins, or molecules exerting a chemical or pharmaceutical effect. Optionally such molecules or moieties are reversibly or releasably coupled to magnetically susceptible particles.

The present invention is also based on the observation that the magnetic field above a Halbach array is not uniform (see FIG. 4a). The magnetic field above the Halbach array has zones where the magnetic flux density and/or gradient is significantly higher than the immediately surrounding field. By providing methods to map this field and manufacture apparatus for positioning a cell in these zones, the present invention permits the skilled person to deliver magnetically susceptible particles into cells with high efficiency.

In one aspect the present invention provides a method of delivering a reagent into a cell, the method comprising positioning at least one cell, and at least one magnetically susceptible particle attached to the reagent, in the magnetic field of a Halbach array such that the magnetically susceptible particle is attracted to and contacts the cell.

In some embodiments the method preferably further comprises the step of oscillating the magnetic field. The direction of oscillation may be substantially perpendicular to the direction of attraction of the magnetically susceptible particles to the Halbach array. In some embodiments oscillation of the magnetic field is achieved by applying an oscillating movement to the Halbach array.

Cell(s) and magnetically susceptible particle(s) are preferably positioned at one or a plurality of discrete addresses formed on a support. The method may comprise aligning at least one of said discrete addresses with a zone of highest magnetic flux density and/or gradient of the Halbach array. More preferably the method comprises aligning at least two of said discrete addresses within one or more zones of highest magnetic flux density and/or gradient of the Halbach array. The alignment may lead to the discrete addresses being respectively aligned with several zones. Each address will normally only be aligned with one zone but each zone may be aligned with one address or with two or more (e.g. several) addresses.

Accordingly the present invention provides a method of delivering a reagent into a cell having the steps of: (i) providing a magnetically susceptible particle comprising the reagent; (ii) providing a Halbach array for exerting a magnetic force on the particle; and (iii) positioning the particle and cell within the array's force field such that the magnetic force urges the particle against the cell.

Step (ii) may optionally involve determining the zones of highest magnetic flux density and/or gradient within the array's force field; and step (iii) may optionally involve positioning the cell in one of the zones of highest magnetic flux density and/or gradient.

The reagent maybe chosen from the group consisting of: an oligonucleotide, DNA, RNA, RNAi, siRNA, an aptamer, DNA encoding a gene of interest, a nucleic acid expression construct, an amino acid, a peptide, a peptide mimetic, a protein, an antibody, an antibody fragment, an scFv, a pharmaceutical, a carbohydrate, a fatty acid or small molecule. The reagent may be a therapeutic agent. In some embodiments the reagent is a nucleic acid and the method results in the genetic transformation (transfection) of the target cell.

In some embodiments the method is performed in vitro. In some other embodiments the cell is a cell in situ in the body of an animal.

In another aspect of the present invention apparatus for the delivery of a reagent into a cell is provided, the apparatus comprising:
i) a Halbach array of magnets; and
ii) a support for positioning cells in the magnetic field of the Halbach array.

In some preferred embodiments the apparatus further comprises means to oscillate the magnetic field of the Halbach array.

In use, the apparatus may further comprise at least one cell positioned on the surface of the support and at least one magnetically susceptible particle attached to a reagent applied to the support such that it is capable of contacting said cell, wherein the magnetic field of the Halbach array is configured to attract said magnetically susceptible particle(s) towards said surface.

One or a plurality of discrete addresses may be provided on the support (as described for the method above), the support and Halbach array being mutually configured in the apparatus to align at least one of said discrete addresses with a zone of highest magnetic flux density and/or gradient of the Halbach array in order to maximize the force on said particle. Optionally at least two of said discrete addresses are aligned within one or more zones of highest magnetic flux density and/or gradient of the Halbach array. Alignment may lead to the discrete addresses being respectively aligned with several zones. Each address will normally only be aligned with one zone but each zone may be aligned with one address or with two or more (e.g. several) addresses. Cells and particles are preferably positioned at each discrete address. In some preferred embodiments the support is a multi-well plate and the discrete addresses are formed by selected individual wells of the plate.

In a further aspect of the present invention a method of manufacturing an apparatus for the magnetic delivery of a reagent into a cell is provided, the apparatus having a Halbach array and a support for positioning cells in the magnetic field of the Halbach array, the method comprising:
(a) providing a Halbach array;
(b) mapping the magnetic flux density and/or gradient of the magnetic field of the Halbach array;
(c) providing a cell support having one or a plurality of discrete addresses spatially configured to align with zones of highest flux density and/or gradient of the Halbach array when the support is assembled in the apparatus;
(d) assembling the Halbach array and support to provide an apparatus for the magnetic delivery of a reagent into a cell.

In another aspect of the present invention a magnetically susceptible particle attached to a reagent is provided for use in a method of treatment, the treatment comprising delivering the reagent into a cell or cells of an animal or human subject by a method comprising administering the magnetically susceptible particle to a tissue in the subject where treatment is required, positioning the tissue and magnetically susceptible particle in the magnetic field of a Halbach array such that the magnetically susceptible particle is attracted to and contacts cells of said tissue.

In a further aspect of the present invention the use of a magnetically susceptible particle attached to a reagent in the manufacture of a medicament for the treatment of a disease is provided, the treatment comprising delivering the reagent into a cell or cells of an animal or human subject by a method comprising administering the magnetically susceptible particle to a tissue in the subject where treatment is required, positioning the tissue and magnetically susceptible particle in the magnetic field of a Halbach array such that the magnetically susceptible particle is attracted to and contacts cells of said tissue.

In yet a further aspect of the present invention a method of treatment of a human or animal in need of treatment is provided, the method comprising delivering a reagent into a cell of an animal or human subject and having the steps of:
(i) administering a magnetically susceptible particle attached to the reagent to a tissue in the subject where treatment is required; and
(ii) positioning the tissue and magnetically susceptible particle in the magnetic field of a Halbach array such that the magnetically susceptible particle is attracted to and contacts cells of said tissue.

In some embodiments of the therapeutic uses and methods of treatment the treatment further comprises the step of oscillating the magnetic field. The therapeutic uses and methods of treatment may further comprise the step of aligning the tissue with at least one zone of highest magnetic flux density and/or gradient of the Halbach array in order to maximize the force applied to the particle(s). For example, the treatment may comprise the steps of immobilizing the subject relative to the Halbach array and aligning the subject such that at least a portion of the tissue of interest is aligned with at least one of the zones of highest magnetic flux density and/or gradient of the Halbach array.

These and further aspects of the invention are described in more detail herein below.

The invention is described with reference to the following figures and examples.

### DESCRIPTION OF THE DRAWINGS

FIG. 1

A: An example of the arrangement of magnets in a Halbach array

B: An illustration of the ‘one sided flux’ generated by the array

C: Examples of magnetic flux patterns from a Halbach array. The unusual flux trapping results in a high gradient on the top surface.

FIG. 2

Transfection efficiency (based on luciferase fluorescence) of the Halbach system compared to other agents. Expression levels of luciferase (expressed in RLUs) are shown for controls (“Con” no DNA); cells with DNA only added (“DNA”); cells transfected with Lipofectamine2000™ (“LF2000”); Polymag® plus DNA but with no magnetic field applied (“PM”); Polymag® plus DNA with a standard, static array of NdFeB magnets (“Static”); and Polymag® plus DNA using the Halbach array (“Halbach”).

FIG. 3

Luciferase activity in NCI-H292 human lung epithelial cells transfected with pCIKLux luciferase reporter construct using OzBiosciences Polymag® particles with “standard” and Halbach arrays as well as naked DNA controls. Transfections were performed in 96 well tissue culture plates using 0.1 μg DNA/well with 2 hours transfection time.

FIG. 4

(a) Flux variation in the x-y plane of the Halbach transfection array at 3 mm above the array surface (level of cell transfection). (b) Average variation in transfection efficiency along the x-axis (N=4 wells in the y-axis per point) as assayed by luciferase fluorescence.

FIG. 5

Fluorescence intensity vs. position in the x-y plane relative to the Halbach array for all transfected wells within the 96-well plate. Z position is 3 mm above the Halbach array.

FIG. 6

A histogram comparing luciferase activity in HEK293 T cells transfected with either a static or oscillating magnetic array. 150 nm magnetic nanoparticles coated with pCIKLux luciferase reporter were used in both cases.

FIG. 7

A histogram showing luciferase activity in NCI-H292 human lung epithelial cells transfected with OzBiosciences Polymag® particles coated with pCIKLux luciferase reporter construct in response to static and oscillating magnetic fields. All transfections were performed in 96 well tissue culture plates using 0.1 μg DNA/well. Genejuice (GJ) and Lipofectamine 2000 (LF2000) transfections were carried out according to the manufacturer's recommended protocol. Data shown as mean±SEM (n=6 for all groups). Magnet diameter=6 mm.

### DETAILED DESCRIPTION OF THE INVENTION

The present invention concerns the use of a magnetic field to place a magnetically susceptible particle comprising a reagent in contact with a cell in order to deliver the particle into the cell. This may be achieved by placing the cell between a magnetically susceptible particle and a magnetic field source. This arrangement results in the particle being drawn toward the magnetic field source and, thereby, into contact with the cell. Thus the present invention provides methods for delivering a reagent into a cell comprising the steps of: i) providing a cell and a magnetically susceptible particle comprising the reagent; and ii) applying a magnetic field such that said particle is drawn towards and contacts said cell.

Some, but not all, aspects of the present invention also concern the use of oscillating magnetic fields to deliver the magnetically susceptible particle(s) into the cell(s). It has been observed that the use of an oscillating magnetic field increases the efficiency with which the magnetically susceptible particles are delivered into the cell. Without the present invention being bound or limited by theory, it is believed that the increase in efficiency is due to the oscillating field moving the magnetically susceptible particle repeatedly across the surface of the cell, a process thought to stimulate the uptake of the particles by endocytic cellular processes. Thus the present invention further provides methods for delivering a reagent into a cell comprising the steps of: i) providing a cell and a magnetically susceptible particle comprising the reagent; and ii) applying a magnetic field such that said particle is drawn towards and contacts said cell; and further comprising the step of iii) oscillating the magnetic field.

In one aspect, the magnetic field may be either static, oscillating, or may be alternated between static and oscillating modes. A magnetically susceptible particle will be drawn towards the source of either a static or magnetic field. Therefore a static or oscillating magnetic field may be used to place the particle in contact with the cell. Subsequently, in some embodiments the magnetic field may either continue, or start, oscillating in order to move the particle across the surface of the cell.

The frequency and amplitude with which the magnetic field is oscillated affects the efficiency with which the particles are delivered into the cell. At high frequencies of oscillation, such as greater than 3 kHz, or greater than 5 kHz, for example greater than 10 kHz, the particles will experience a substantial heating effect due to hysteresis and eddy current effects. Such heating of the particle may be toxic to any cell with which it is in contact. Consequently, the frequency of oscillation should be kept within a suitable range such as up to (i.e. no more than) 3 kHz, or up to 1 kHz or up to 100 Hz, for example up to 10 Hz or up to 2 Hz. In one aspect the field oscillates with a frequency of from 0 to 100 Hz such as from 1 mHz to 10 Hz or from 500 mHz to 5 Hz, for example 1 to 3 Hz or 2 Hz.

The amplitude of the magnetic field oscillation affects the extent to which gradients in the magnetic field are moved past the magnetically susceptible particle, and therefore affects the forces acting on the particle. In one embodiment the amplitude of the oscillation is from 0 to 5000 μm, such as 10 to 2000 μm or 20 to 1000 μm, for example 50 to 500 μm or 100 to 300 μm. The amplitude of oscillation may be 200 μm. In other embodiments the amplitude of oscillation is up to (i.e. no more than) 5000 μm, such as up to 2000 μm or up to 1000 μm, for example up to 500 μm or up to 200 μm.

The inventors believe that the efficiency with which the magnetically susceptible particles are delivered into the cell increases as the flux density and/or gradient of the magnetic field increases. A magnetic field with a higher magnetic flux density and/or gradient exerts a greater magnetic force on a magnetically susceptible particle, and it is thought that this greater force on the particle enhances the efficiency of delivering the particle into the cell. Magnetic field sources that generate fields with high flux densities and/or gradients are therefore desired for use in the present invention. Such fields may be produced by one or more strongly magnetized permanent magnets (e.g. an array or magnets), or one or more electromagnets having a large number of coil turns and/or carrying a high current.

The strength of permanent magnets is limited by the physical properties of their constituents and their size. The strength of an electromagnet is limited by the heating effects of high turn number coils, or those carrying large currents. These problems can be ameliorated by using very-low resistance conductive material, but such materials may be expensive and/or require cooling.

Where a strong permanent magnet is required, it is common to use rare-earth element magnets such as NdFeB magnets which have very high magnetic flux densities and gradients relative to their mass. For a given size, the maximum attainable flux density and/or gradient is limited by the magnet's physical properties.

The maximum flux density and/or gradient produced by a given permanent magnet type can be increased by arranging individual bipolar magnets into arrays known as Halbach arrays. These Halbach arrays produce a magnetic field that is diminished on one side of the array and augmented on the opposite side of the array. In this manner the flux density and/or gradient on the augmented side of the array can be much higher than a conventional non-Halbach array (for example, the field produced by the augmented side may be approximately twice the strength of that obtained by a conventional array). Individual bipolar electromagnets may also be arranged into a Halbach array to produce an augmented ‘one-sided’ magnetic field. Accordingly, the present invention concerns the use of Halbach arrays, of any type, to deliver a reagent into a cell. Thus the present invention provides methods for delivering a reagent into a cell comprising the steps of: i) providing a cell and a magnetically susceptible particle comprising the reagent; and ii) applying a magnetic field such that said particle is drawn towards and contacts said cell; wherein the magnetic field is produced by a Halbach array. In a further aspect, the magnetic field produced by the Halbach array may be oscillated.

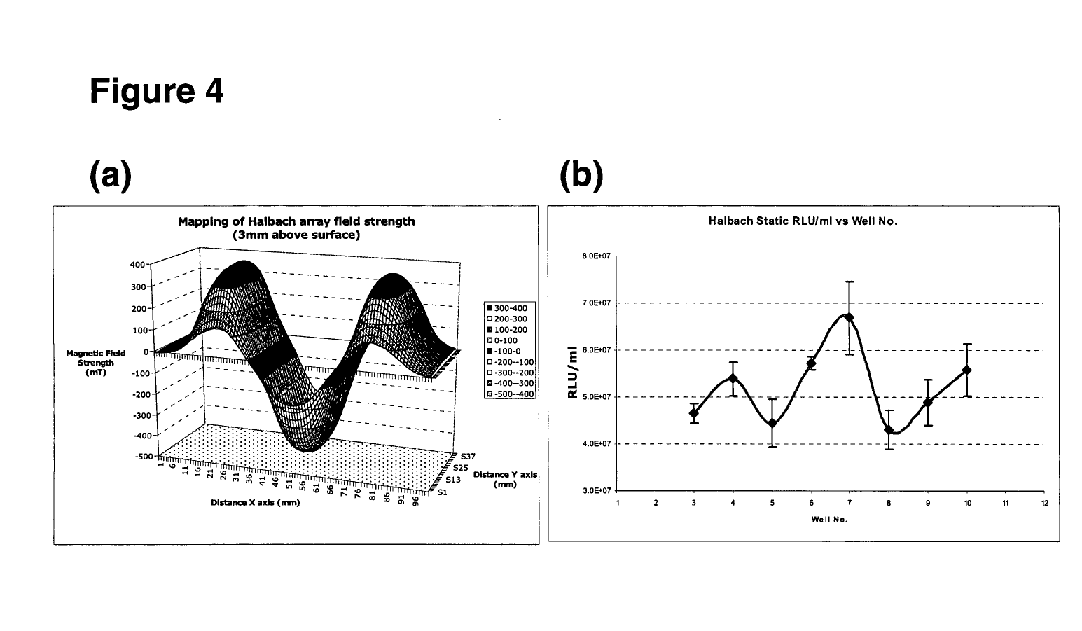

The flux density and/or gradient produced by a Halbach array may not be uniform. For example, the field above a planar Halbach array varies in both the x and y axes (see FIG. 4a). The variation in the magnetic field produced by a Halbach array may lead to a variation in the efficiency of delivering particles into cells located in different regions of said field. It has been observed that the efficiency of delivering particles into cells is highest in the zones of highest magnetic flux density and/or gradient. The present invention is concerned with identifying said zones of highest magnetic flux density and/or gradient and positioning cells within those zones. Accordingly, the present invention provides methods for delivering a reagent into a cell comprising providing at least one cell and at least one magnetically susceptible particle comprising the reagent and applying a magnetic field such that said particle is drawn towards and contacts said cell, wherein the cell and magnetically susceptible particle are positioned within the zones of highest magnetic flux density and/or gradient. In one aspect the field produced by the Halbach array may be oscillated. The oscillation may be such that the positions of the zones of highest magnetic field density and/or gradient are oscillated.

In one aspect, the position of the Halbach array may be shifted in either of the x or y axis of a plane, or alternately along both axes, such that the locations of the zones of highest magnetic flux density and/or gradient are shifted from their original position. The Halbach array may be held stationary in the new position for a period of time up to 24 hrs, such as up to 2 hrs, or up to 1 hr or up to 30 minutes, for example, up to 20 minutes. The movement of the Halbach array may then be repeated as desired so that the zones of highest magnetic flux density and/or gradient are tracked across the majority of a desired area (for example more than 50% of a desired area, such as more than 60%, or more than 70%, or more than 80% of a desired area). The desired area may be the support for positioning the cells (e.g. the area of a multi well plate). In aspects where the magnetic field is oscillated, the centre of oscillation of each zone of highest magnetic flux density and/or gradient may be shifted as described above such that the centres of oscillation are tracked across the majority of a desired area.

The magnetic field surrounding a magnetic field source may be mapped using a magnetometer. Following the mapping of the magnetic field, a support may be provided for supporting the cells within the zones of highest magnetic flux density and/or gradient. In one aspect the support forms a plurality of discrete addresses, wherein the addresses coincide with the zones of highest magnetic flux density and/or gradient. Thus the invention provides a method wherein the cells and magnetically susceptible particles are positioned at one or a plurality of discrete addresses that coincide with the zones of highest magnetic flux density and/or gradient.

The methods of the present invention can be employed in vitro or in vivo. The term “in vitro” is intended to encompass experiments with cells in culture whereas the term “in vivo” is intended to encompass experiments with intact multi-cellular organisms.

Regarding in vivo embodiments, the present invention provides for the use of a magnetically susceptible particle attached to a reagent in the manufacture of a medicament for the treatment of a disease, the treatment comprising delivering the reagent into a cell or cells of an animal or human subject by a method comprising administering the magnetically susceptible particle to a tissue in the subject where treatment is required, positioning the tissue and magnetically susceptible particle in the magnetic field of a Halbach array such that the magnetically susceptible particle is attracted to and contacts cells of said tissue.

The present invention also provides at least one magnetically susceptible particle attached to a reagent for use in a method of treatment, the treatment comprising delivering the reagent into a cell or cells of an animal or human subject by a method comprising administering the magnetically susceptible particle to a tissue in the subject where treatment is required, positioning the tissue and magnetically susceptible particle in the magnetic field of a Halbach array such that the magnetically susceptible particle is attracted to and contacts cells of said tissue. The treatment may also comprise aligning the cells of said tissue with one or several zones of highest magnetic flux density and/or gradient of the Halbach array in order to maximize the force applied to the particles. For example, the treatment may comprise the steps of immobilizing the subject relative to the Halbach array and aligning the subject such that at least a portion of the tissue of interest is aligned with at least one of the zones of highest magnetic flux density and/or gradient of the Halbach array.

The present invention also provides a method of treatment of a human or animal in need of treatment, the method comprising delivering a reagent into a cell of an animal or human subject and having the steps of:
(i) administering a magnetically susceptible particle attached to the reagent to a tissue in the subject where treatment is required; and
(ii) positioning the tissue and magnetically susceptible particle in the magnetic field of a Halbach array such that the magnetically susceptible particle is attracted to and contacts said cells of said tissue.

The treatment methods may further comprise oscillating said magnetic field, as described herein.

The present invention may find use in the treatment of a wide range of diseases and conditions. Treatment may be effected by delivery of a therapeutic agent into the target cell(s). A wide range of therapeutic agents (e.g. nucleic acids, peptides, proteins, antibodies and antibody fragments, and small molecule drugs) may be attached to the magnetically susceptible particles. Treatments may involve gene therapy, i.e. transfection of cells with nucleic acid encoding a gene.

The subject to be treated may be any animal or human. The subject is preferably mammalian, more preferably human. The subject may be a non-human mammal, but is more preferably human. The subject may be male or female. The subject may be a patient.

The present invention also provides apparatus for the delivery of a reagent into a cell, the apparatus comprising: i) a Halbach array of magnets; and ii) a support for positioning cells in the magnetic field of the Halbach array. In one aspect the apparatus further comprises means to oscillate the Halbach array.

In another aspect, the apparatus further comprises at least one cell positioned on the surface of the support and at least one magnetically susceptible particle applied to the support such that it is capable of contacting said cell, wherein the magnetic field of the Halbach array is configured to attract said magnetically susceptible particle(s) towards said surface.

In a further aspect the support has a plurality of discrete addresses, wherein the addresses coincide with the zones of highest magnetic flux density within the array's magnetic field. Preferably each address is configured to retain and support at least one cell into which reagent is to be delivered and to allow magnetically susceptible particles to contact the cell. For example, the address may be a well in a multi-well plate, or a region of the support providing a substrate suitable for cell attachment, e.g. treated so as to allow adherence and/or culture of cells.

In yet a further aspect the apparatus comprises a support comprising a multi-well plate, the plate having one or a plurality of wells (preferably several, e.g. 2 or more, 5 or more, 10 or more, 20 or more, 30 or more, 40 or more, 50 or more, 60 or more, 70 or more, 80 or more, 90 or more, optionally less than 100, or optionally less than 400) configured in the apparatus to align with the zones of highest flux density and/or gradient of the Halbach array. In a related aspect, the apparatus may comprise cells and/or magnetic particles held in the aligned wells.

In a further aspect of the present invention a method is provided for the manufacture and/or production of an apparatus that is suitable for the magnetic delivery of a reagent into a cell, such apparatus being in accordance with the apparatus and methods described herein, the method of production comprising: (a) providing a Halbach array; (b) mapping the magnetic flux density and/or gradient of the magnetic field of the Halbach array; (c) producing a cell support having one or a plurality of discrete addresses spatially configured to align with zones of highest flux density and/or gradient of the Halbach array when the support is assembled in the apparatus; (d) assembling the Halbach array and support to provide an apparatus that is suitable for the magnetic delivery of a reagent into a cell.

The information obtained from the mapping step (b) is preferably used to design the cell support such that when assembled in the apparatus the support has addresses that are positioned in the zones of highest flux density and/or gradient of the Halbach array.

The spatial configuration of addresses on the support may include consideration of the three-dimensional (x, y and z axis positions) of the surface of the support, such surface optionally providing a location for cell attachment (e.g. a cell culture substrate). The method may therefore also comprise a step of modeling the three-dimensional magnetic flux density and/or magnetic field gradient of the Halbach array and designing a support that, when positioned at a predetermined spacing from said Halbach array, has one or a plurality of discrete cell culture substrate addresses positioned in the zones of highest flux density and/or gradient of the Halbach array.

The Halbach array may be incorporated into a convenient stand or base such that cell-containing receptacles, particularly conventional labware such as 6-, 24-, 96-, 192- or 384-well plates, tissue culture flasks or dishes, can be supported in an orientation appropriate to the position and type of cells to be transfected. For adherent cells growing on the bottom of wells or flasks this may be achieved by incorporating the array into an essentially flat base on which the container rests, optionally with one or more shaped recesses to retain the plates or flasks more securely. Other arrangements, such as bases comprising holes designed to receive standard sized ‘Eppendorf’-type tubes are also possible. The body of the base or stand may conveniently take the form of a non-magnetic block formed from any suitable material, such as a polymer or plastics material.

Cells are cultured in appropriate media in flasks or multi-well plates, which are then placed onto the array. Magnetic nanoparticles carrying DNA or other reagent are introduced to the culture before or afterwards and the high-gradient field increases sedimentation of the particle/reagent complex, rapidly pulling it into contact with the cells. This type of array increases the available magnetic field gradient and force on the particles by up to 25-fold as compared to commercially available conventional devices and the increased force significantly improves both transfection time and efficiency.

The invention also provides a device for use in magnetic transfection of cells, said device comprising magnets, and characterised in that said magnets are arranged in a Halbach array.

Said magnets may be housed in a base capable of supporting a cell-containing receptacle, and may comprise an essentially flat supporting surface with a recess capable of receiving a cell-containing receptacle.

In one embodiment, the base comprises a non-magnetic block comprising said magnets. The block may be, for example, a block of a polymer or plastics material or a non-magnetic metal (such as aluminium).

The magnets may be permanent magnets, such as rare earth magnets, for example neodymium-iron-boron (NdFeB) permanent magnets. In one aspect the array provides a field strength, measured 1 cm from the proximal surface (by which is meant the surface closest to or in contact with the cell-containing receptacle when in use) is 10 mT or greater, such as 100 mT or greater. In another aspect it is 130 mT or greater.

In another aspect, the invention provides an apparatus comprising the device as described, together with a receptacle suitable for containing cells. Said receptacle referred to above may be a conventional multi-well plate such as a 6-well, 12-well, 24-well, 96-well, 192-well or 384-well plate. Alternatively it is a tissue culture flask or a dish, such as a standard 35 mm diameter dish or Petri dish. The apparatus, when in use, may be capable of delivering a field strength of more than 10 mT, such as more than 100 mT, or more than 200 mT, or more than 300 mT, for example more than 400 mT to cells, as measured at the cell surface.

In a further aspect, the invention provides a method of transfecting cells comprising exposing said cells to magnetisable particles coupled to one or more nucleic acid or polypeptide molecules and positioning said cells within the magnetic field generated by a Halbach array. The field strength to which the cells are exposed may be 10 mT or greater, such as 100 mT or greater, for example greater than 130 mT, or 300 mT or greater, for example 400 mT or greater.

In a further aspect, the invention provides a kit comprising the above-described device or apparatus, together with magnetisable particles capable of being coupled to a molecule or moiety, preferably a nucleic acid or polypeptide molecule, for transfection. Optionally such a kit may include coupling reagents, buffers, and control reagents.

Reagent

As used herein, a ‘reagent’ refers to an agent performing a desired function within a cell. The reagent may function as a marker of a particular cellular process or structure, or may modulate a cellular process or function. In one aspect the reagent may specifically bind to a cellular target molecule. For example, the reagent may be an inhibitor or an activator of a cellular process such as protein or DNA synthesis, protein transport, respiration or a particular metabolic pathway.

Reagents may be any pharmaceutical compound, molecule derived from a biological source, or artificially synthesized molecule. In one aspect, the reagent may comprise a nucleotide or polynucleotide such as DNA, RNA, interfering RNAs (e.g. RNAi or siRNA), nucleotide analogs, polynucleotide analogs or aptamers. In another aspect, the reagent may comprise an amino acid or peptide such as a polypeptide, amino acid analog, peptide mimetic, antibody, antibody fragment (e.g. single chain antibody), or scFv. In a further aspect the reagent may be an organic or inorganic compound, such as a heterorganic or organometallic compound, or a salt, ester or other pharmaceutically acceptable form of the compound. Typically such compounds will have a molecular weight up to 10,000 grams per mole (g/mol), such as up to 500 g/mol, for example up to 1000 g/mol or up to 500 g/mol. In a yet further aspect, the reagent may be a therapeutic agent which may have an activity useful in the diagnosis, prevention or treatment of a disease, disease state or clinical disorder.

In one aspect of the present invention the reagent is used in a method of transfection i.e. the introduction of foreign material into the cell. In one aspect the reagent is a nucleic acid or nucleic acid analogue such as DNA or RNA. The nucleic acid or nucleic acid analogue may encode a protein or functional protein fragment implicated in a disease state. Alternatively, said protein or functional protein fragment may be directly transfected into the cell. In another aspect, the absence or deficiency of the said protein or functional protein fragment from the cell contributes to a disease state (e.g. the Cystic Fibrosis CFTR-1 membrane protein).

In some embodiments the reagent is nucleic acid encoding a gene, preferably operably linked to a control sequence (e.g. a promoter) and optionally to other control sequences, e.g. enhancers and/or polyA sequences, to provide an expression construct useful in gene therapy applications. For example, the gene may be the wild type CFTR-1 membrane protein operably linked to a mammalian (e.g. human) promoter. Such a construct may be useful in the treatment of cystic fibrosis by gene therapy. As shown by the examples below, human lung epithelial cells may be transfected using the methods and apparatus of the present invention.

Cell

As used herein, ‘cell’ is a term used to refer to a cell that it is desired to deliver the reagent into. It may be referred to as a target cell. The cell may be any cell, for example a bacterial, protozoan, fungal, plant or animal cell. In one aspect the cell may be a mammalian cell, such as a lung cell, kidney cell, nerve cell, mesenchymal cell, muscle cell, liver cell, erythrocyte, white blood cell, pancreatic cell, epithelial cell, endothelial cell, bone cell, skin cell, gastrointestinal cell, bladder cell, uterine cell, endocrine cell, prostate cell, stem cell, culture line cell or tumour cell. The cell may be a non-human mammalian cell, for example rabbit, guinea pig, rat, mouse or other rodent (including cells from any animal in the order Rodentia), cat, dog, pig, sheep, goat, cattle, horse, non-human primate or other non-human vertebrate organism. Alternatively, the cell may be a human cell. In vitro methods may involve cells in culture. In vivo methods may involve cells in situ in the human or animal body.

The cell may be an isolated cell not associated with other cells, or may form part of a tissue or organ. The cell may be either in vitro or in vivo. In one aspect the cell forms part of an adherent cell layer, such as the cell layers typically grown on the base of cell culture flasks.

Mixture

Reagents and cells may be provided such that they are able to contact each other. Such an arrangement is generally referred to as a mixture which includes cells and reagents Cells and reagents may both be provided suspended in solution, e.g. in culture media. In some embodiments cells are adhered to a support. In such circumstances the reagent may be contained in liquid or fluid (e.g. culture media) bathing the cells.

Magnetically Susceptible Particle

Magnetically susceptible particles can include magnetically susceptible particles, magnetisable particles or particles that can be manipulated (e.g. moved) and/or positioned by a magnetic field. The magnetically susceptible particles can be non-magnetic but susceptible to manipulation or positioning by a magnetic field, or be magnetic (e.g. a source of a magnetic field lines).

Typically the particles are of a size suitable to deliver the reagent into the cell without causing damage to the cell. In one aspect, the particles have a mean size of between 10 μm and 5 nm, such as between 1 μm and 10 nm, for example between 200 nm and 100 nm. In another aspect the magnetically susceptible particles may be spherical beads and may have a diameter of at least about 0.05 microns, at least about 1 micron, at least about 2.5 microns, and typically less than about 20 μm.

Not wishing to be limited by theory, it is believed that larger particles will give improved uptake. For example, for magnetite particles >30 nm will experience a torque in an oscillating magnetic field as dictated by the formula:

τ=μB sin θ

where t is the torque, μ is the magnetic moment, B is the magnetic flux density and θ is the angle between the applied field and the particle's magnetization vector. For example, the precise amount of torque is influenced the particles shape. The movement of the particle induced by this torque is believed to ‘drag’ the particle into and across the surface of the cell, inducing uptake of the particle by an undetermined endocytic mechanism. The uptake of the particle by normal cellular processes means that there is no mechanical damage to the cell (as compared to, for example, biolistic methods or electroporation), thus improving the rate of cellular survival post particle delivery.

A magnetically susceptible particle can be, for example, a magnetically susceptible particle described, in U.S. Patent Application Publication Nos. 20050147963 or 20050100930, or U.S. Pat. No. 5,348,876, each of which is incorporated by reference in its entirety, or commercially available beads, for example, those produced by Dynal AS (Invitrogen Corporation, Carlsbad, Calif. USA) under the trade name DYNABEADS™ and/or MYONE™. In particular, antibodies linked to magnetically susceptible particles are described in, for example, United States Patent Application Nos. 20050149169, 20050148096, 20050142549, 20050074748, 20050148096, 20050106652, and 20050100930, and U.S. Pat. No. 5,348,876, each of which is incorporated by reference in its entirety.

In one aspect the particle comprises a paramagnetic, superparamagnetic, ferromagnetic and/or anitferromagnetic material, such as elemental iron, chromium, manganese, cobalt, nickel, or a compound and/or a combination thereof (e.g. manganese and cobalt ferrites). For example, suitable compounds include iron salts such as magnetite (Fe₃O₄), maghemite (γFe₂O₃), greigite (Fe₃S₄) and chromium dioxide (CrO₂).

The particles may comprise the magnetic material embedded in a polymer, for example within the pores of a polymer matrix. Alternatively, the particles may comprise a magnetic core surrounded by a biocompatible coating, for example silica or a polymer such as dextran, polyvinyl alcohol or polyethylenimine.

The magnetically susceptible particle comprises a reagent. The reagent may be associated with (e.g. conjugated to) the particle by covalent or non-covalent bonds (for example, hydrogen bonding, electrostatic interactions, ionic bonding, lipophillic interactions or van der Waals forces). In one aspect the reagent and particle are covalently linked, for example by exposing the reagent to particles bearing reactive side chains, for example benzidine for linking to the tyrosine residues of proteinaceous reagent, or periodate for linking to carbohydrate groups. In another aspect the particle may be linked to a molecule with binding activity (e.g. avidin) and the reagent may be linked to a ligand of said binding molecule (e.g. biotin). This enables the particle and reagent to be easily conjugated in vitro. In a further aspect the particle may comprise the reagent absorbed into a matrix, such as a polymer matrix.

Halbach Array

As used herein, the term ‘Halbach array’ is used to describe an array of dipole magnets arranged with their poles in a specific sequence of orientations such that there is an augmentation of the magnetic field on one side of the array and a reduction of the magnetic field on the opposite side of the array relative to a conventional array (i.e. an otherwise identical array of magnets with dipoles arranged in a different, non-Halbach, sequence). This effect was first noted by John Mallinson [Mallinson, 1973, IEEE Transactions on Magnetics 9: 678] and was subsequently published by Klaus Halbach [K. Halbach, 1981, Nucl. Inst. and Methods 187. pp. 109-117]. The dipole magnets may be permanent magnets or electromagnets. In one aspect the magnets are NdFeB permanent magnets.

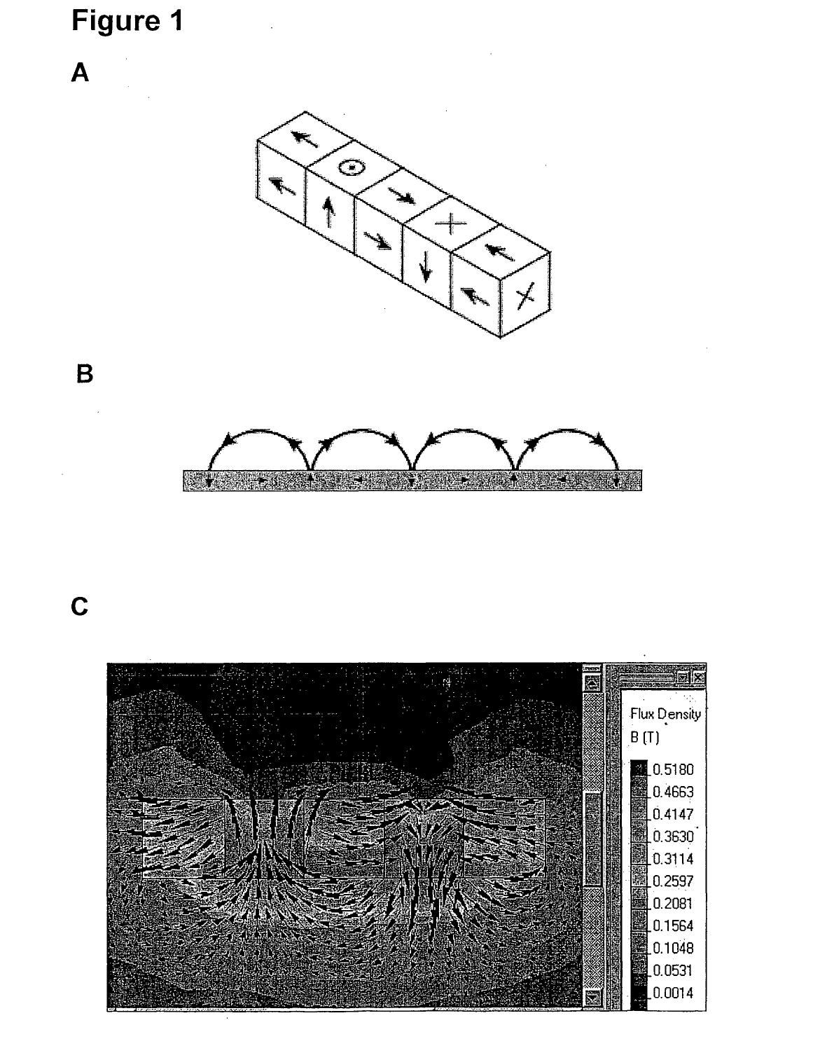

An example of a simple Halbach array showing the orientations of the constituent dipole magnets is shown in FIG. 1A. The way in which the flux lines of the constituent dipole magnets add to give a ‘one-sided’ flux is illustrated in FIG. 1B. The Halbach array may be of any size sufficient to generate a field of the required shape and size. In one aspect the Halbach array comprises an array of no more than 9 by 12 dipole magnets, such as no more than 6 by 8 dipole magnets, for example 3 by 4 dipole magnets. In another aspect the Halbach array comprises a linear array of 3 to 5 dipole magnets.

In a planar Halbach array the x and y components of the magnetic flux are in phase above the plane, and π/2 out of phase below the plane of the array; as such, in the ideal case of an array of infinite length, the magnetic flux on one face of the array is doubled and the flux on the opposite face of the array is cancelled. In arrays of finite length some stray field is produced on the opposite face, though the field remains highly asymmetric (See FIG. 1C), with the augmented face having approximately double the flux density of a conventional array.

In addition to the augmented field strength as compared to a conventional array, the field above the augmented face of the array is also highly contained; this produces a high field gradient.

In addition to planar arrays, Halbach arrays can be arranged into cylindrical or spherical arrays. In these aspects, the constituent dipole magnets may be arranged to give a ‘one-sided field’ that is augmented on either the inner or outer face of the cylinder or sphere. In one particular aspect, Halbach cylinders can be arranged to have a bipolar uniform magnetic field within the bore of the cylinder. Alternatively, the constituent dipole magnets may be arranged to give a quadripolar field within the cylinder bore.

Zones of Highest Magnetic Flux Density and/or Gradient

As used herein, ‘zones of highest magnetic flux density and/or gradient’ is used to mean the zones in the magnetic field above the augmented face of the Halbach array that have a flux density and/or gradient that is significantly above that of the immediately surrounding field. The zones may correspond to zones where the field strength measured at 3 mm above the arrays surface is over 200 mT, such as over 300 mT, for example over 400 mT at 3 mm above the array surface. Alternatively, the zones may correspond to zones where the magnetic field gradient measured at 3 mm above the array surface is greater than 30 mT/mm, such as greater than 40 mT/mm, for example greater than 50 mT/mm, 60 mT/mm, 70 mT/mm or 80 mT/mm. In another aspect, the zones of highest magnetic flux density and/or gradient are zones having magnetic flux density and/or gradient within 30% of the maximum magnetic flux density and/or gradient provided by the Halbach array. For example, the zones may have magnetic flux density and/or gradient within 25% of the maximum magnetic flux density and/or gradient, or within 20% of the maximum magnetic flux density and/or gradient provided by the Halbach array. For example, in the case of within 20% of the maximum magnetic flux density and/or gradient, if the maximum value is 100, the zone will have a value of 80 or more. More preferably the zones have magnetic flux density and/or gradient within one of 10% of the maximum flux density and/or gradient, 5% of the maximum flux density and/or gradient, 3% of the maximum flux density and/or gradient, 2% of the maximum flux density and/or gradient, and/or 1% of the maximum flux density and/or gradient.

The magnetic field over the surface of a Halbach array is non-uniform. For example, at the zones where the flux lines meet the surface of the array (see FIGS. 1B and 4a) the flux is particularly dense and also undergoes a reversal in direction. This leads to particularly high field gradients in these zones. The variation in the magnetic field across the surface of a Halbach array is illustrated in FIG. 4(a) which shows the field above the Halbach array varies in both the x and y axes; the variation in the x axis is particularly pronounced, with the field going from strongly positive to strongly negative and back again.

The location of the zones where the magnetic flux density and/or gradient is highest can be identified when the field above the Halbach array is mapped using, for example, a scanning, 3-axis Hall probe magnetometer such as a Redcliffe Magnetics Magscan 500. Such mapping permits the locations of the zones of highest magnetic flux density and/or gradient to be recorded for subsequent ease of location.

Magnetic Force

As used herein, ‘magnetic force’ means the force that is exerted on a magnetically susceptible particle when it is in a magnetic field having a gradient. The magnetic force may cause the magnetically susceptible particle to move toward the source of the magnetic field. In this case the force is a translational magnetic force. The magnetic force may also cause the particle to experience a torque.

In some arrangements, the magnetic force may cause the particle to move away from the source of the magnetic field. This can occur if the particle is magnetically blocked and unable to rotate.

Force Field

As used herein, the ‘force field’ of a magnet, or of a magnetic array, describes the volume of space surrounding the magnet or magnetic array in which a magnetically susceptible particle will experience a magnetic force.

Support

As used herein, ‘support’ refers to any means for positioning the cell within the flux density of the Halbach array such that, when positioned in the flux by the support the magnetic force on the magnetically susceptible particle urges the particle against the cell.

In one aspect the support positions a cell containing receptacle (such as a tissue culture flask or multiwell plate) above the augmented face of the Halbach array. For example, the support may be a surface for supporting the cell containing receptacle. Alternatively the support may comprise a grip or clamp for holding the cell containing receptacle. In this arrangement any magnetically susceptible particles in the receptacle are drawn down towards the base of the receptacle where they are urged against any cells that may be adhered to the base of the receptacle.

In another aspect the support comprises a recess for receiving a cell containing receptacle. In a further aspect, the support may comprise a plurality of recesses. Each recess may be adapted to support a single cell containing receptacle. Alternatively, each recess may be able to accommodate a plurality of receptacles.

In yet another aspect, the support may be for supporting a mammalian subject within the array force field.

In one aspect the support may have addresses corresponding to the zones of highest magnetic flux density and/or gradient. In some embodiments there may be a plurality of discrete addresses formed on the support for positioning a cell and magnetically susceptible particle, wherein the addresses coincide with the zones of highest magnetic flux density and/or gradient. For example, the support may have marking that allow the cell-containing receptacle to be positioned such that the cell is located in a zone of highest magnetic flux density and/or gradient. Alternatively, the support may have a plurality of recesses each having a unique address, with the addresses corresponding to zones of highest magnetic flux density and/or gradient indicated.

Cell Culture Substrate

As used herein, ‘cell culture substrate’ is used to mean a substrate upon which cells can live and/or grow such as the base of a cell culture flask, or a multi-well plate.

Multi-Well Plate

As used herein, ‘multiwell plate’ refers to a plate having two or more separate wells. The plate may have more than 2 wells, such as 4, 6, 12, 24, 36, 48, 96,192 or 384 wells. The wells may be used to contain and/or culture cells. The wells may have unique addresses for identification of the individual wells. The plates may be disposable.

Surface of the Array

As used herein, ‘surface of the array’ is used to mean the exterior surface of the constituent dipole magnets on the augmented face of the array. In one aspect the cell is positioned no further than 10 mm from the surface of the array, such as no further than 5 mm or no further than 3 mm, for example no further than 2 mm or no further than 1 mm.

Oscillating Magnetic Field

As used herein, ‘oscillating magnetic field’ is used to refer to the movement of the magnetic field. In one aspect the magnetic field causes the particles to move in a first direction toward the Halbach array (the direction of attraction) and the magnetic field oscillates in a second direction at an angle to the first direction. The angle between the first and second directions may be greater than 0° and less than 180°. Preferably it is greater than 0° and less than 90° such as greater than 60° and less than 120° or greater than 70° and less than 110°, for example the angle between the first and second directions may be greater than 80° and less than 100° or greater than 85° and less than 95°. Preferably, the first direction is substantially perpendicular to the second direction.

In one aspect, the magnetic field is oscillated along a single axis. Alternatively, the field may be subjected to planar oscillation relative to the direction of attraction of the magnetically susceptible particle to the Halbach array, e.g. oscillation that is in a plane substantially perpendicular to the direction of attraction. In a further aspect the magnetic field may in addition move into and out of said plane. In a yet further aspect the field may move with a rotational movement.

The magnetic field may oscillate with a frequency up to 3 kHz, such up to 1 kHz or up to 100 Hz, for example up to 10 Hz or up to 2 Hz. In one aspect the field oscillates with a frequency of 0 to 100 Hz such as 1 mHz to 10 Hz or 500 mHz to 5 Hz, for example 1 to 3 Hz or 2 Hz. In other embodiments, the magnetic field may oscillate with a frequency of 0.1 to 3 Hz.

In one aspect the magnetic field is oscillated by physically moving the Halbach array with an oscillating motion. In one aspect the amplitude of the oscillation is between 0 to 5000 μm, such as 10 to 2000 μm or 20 to 1000 μm, for example 50 to 500 μm or 100 to 300 μm. The amplitude of oscillation may be 200 μm. In another aspect the amplitude of oscillation is up to 5000 μm, such as up to 2000 μm or up to 1000 μm, for example up to 500 μm or up to 200 μm.

Alternatively, the magnetic field may be oscillated by oscillating the dipoles of electromagnets comprised within the array. The dipoles of the electromagnets may be made to oscillate by supplying the electromagnet with electrical current of alternating polarity. The current may alternate with a frequency up to 3 kHz, such as up to 1 kHz or up to 100 Hz, for example up to 10 Hz or up to 2 Hz. In one aspect the current oscillates with a frequency of 0 to 100 Hz such as 1 mHz to 10 Hz or 500 mHz to 5 Hz, for example 1 to 3 Hz or 2 Hz. In other embodiments, the current may oscillate with a frequency of 0.1 to 3 Hz.

Genetic Transformation

As used herein, genetic transformation describes the process in which a cell is genetically altered by the uptake, incorporation and expression of exogenous genetic material. The transformation may be temporary or permanent and may or may not be heritable by the progeny of the cell.

Mapping the Magnetic Flux Density

As used herein, mapping the magnetic flux density refers to the process of determining the shape and strength of the magnetic field around the array. The flux density may be mapped using a magnetometer such as a Redcliffe Magnetics Magscan 500. In one aspect the flux density is mapped over a plane above the surface of the array. The mapped plane may be at any selected distance above the surface of the array, such as up to 10 mm or up to 5 mm, for example up to 3 mm or up to 1 mm. By mapping at different heights above the array, it is possible to determine both the magnetic flux density and the magnetic field gradient at various points in three-dimensional space above the array surface. The mapping of the flux density around the array may be used to provide a support that positions the cell in a zone of high flux density and/or gradient. The support may position the cell in a zone of high flux density and/or gradient in a plane at the same distance from the surface of the array as a mapped plane.

Receptacle for Containing Cells/Cell Containing Receptacle

As used herein ‘receptacle for containing cells/cell containing receptacle’ is used to refer to any receptacle or vessel suitable for containing cells, such as a cell-culture flask or dish, multi-well plate, petri dish, test tube, falcon tube or Eppendorf tube. The receptacle may also be suitable for culturing cells.

Means for Oscillating the Halbach Array

As used herein, ‘means for oscillating the Halbach array’ refers to an element that causes the array to oscillate relative to the cell. The element may be a motor, such as an electrical stepper or servo motor. In one aspect the oscillation of the array may be controlled by a computer.

The invention includes the combination of the aspects and preferred features described except where such a combination is clearly impermissible or expressly avoided.

The section headings used herein are for organizational purposes only and are not to be construed as limiting the subject matter described.

Aspects and embodiments of the present invention will now be illustrated, by way of example, with reference to the accompanying figures. Further aspects and embodiments will be apparent to those skilled in the art. All documents mentioned in this text are incorporated herein by reference.

### Examples

### Example 1

In a pilot study, a Halbach array comprising five NdFeB magnets (10×10×25 mm) arranged as in FIG. 1, was placed beneath 2×6 wells of a standard 24-well culture plate containing NCI-H292 (human lung epithelial) cells. The cells were maintained in RPMI 1640 culture media supplemented with 10% foetal calf serum, 100 U/mL penicillin, 0.1 mg/mL streptomycin, 0.25 μg/mL amphortericin-B and 2 mM L-glutamine. Cells were seeded at 5×10³ cells/well in 96 well tissue culture plates and incubated overnight at 37° C. 5% CO₂ to allow the cells to attach. Transfections were performed in SF RPMI medium using Polymag® (OzBiosciences) nanoparticles with 0.1-0.5 μg DNA per well following the manufacturers recommended protocol. Following the addition of reagents, the plates were transferred to an incubator at 37° C. 5% CO₂ and placed above the Halbach array for 20 minutes. At 2 hrs post transfection, the media was replaced with an equal volume of RPMI 1640 culture media supplemented with 10% foetal calf serum, 100 U/mL penicillin, 0.1 mg/mL streptomycin, 0.25 μg/mL amphortericin B and 2 mM L-glutamine. At 48 hrs post transfection, the media was removed from each well and the cells lysed by the addition of 30 μL of cell reporter lysis buffer (Roche). Samples were assayed for luciferase activity using a luciferase assay reagent (Promega) and the total protein concentration determined using a BCA assay reagent (Pierce, USA).

The Halbach array produced a field of 498 mT at the cell surface, while the standard array (5 mm diameter NdFeB magnets) produced a field of 222 mT at the cell surface. The Halbach array also produces a higher field gradient than the standard array, further increasing the forces on the magnetic nanoparticle carriers.

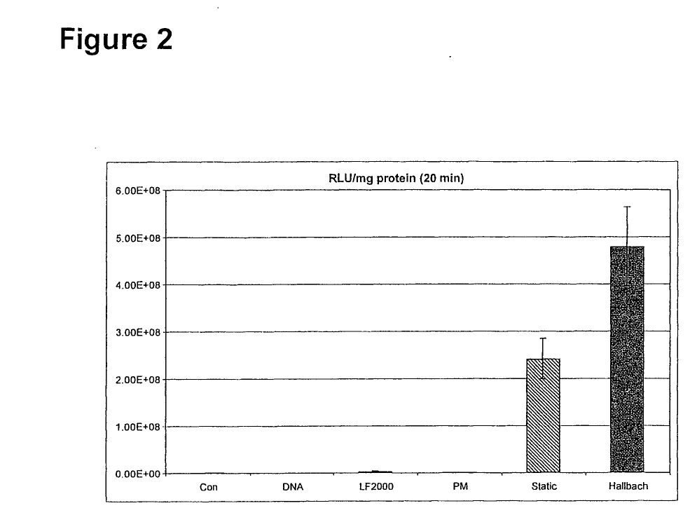

Transfection using the Halbach array has been shown to improve transfection by nearly 2-fold compared to standard magnet arrays and up to 100-fold compared to Lipofectamine2000 (a cationic lipid agent) after 20 minutes (FIG. 2).

### Example 2

Luciferase activity in NCI-H292 was measured in human lung epithelial cells transfected with pCIKLux luciferase reporter construct using OzBiosciences Polymag® particles with “standard” and Halbach arrays as well as naked DNA controls.

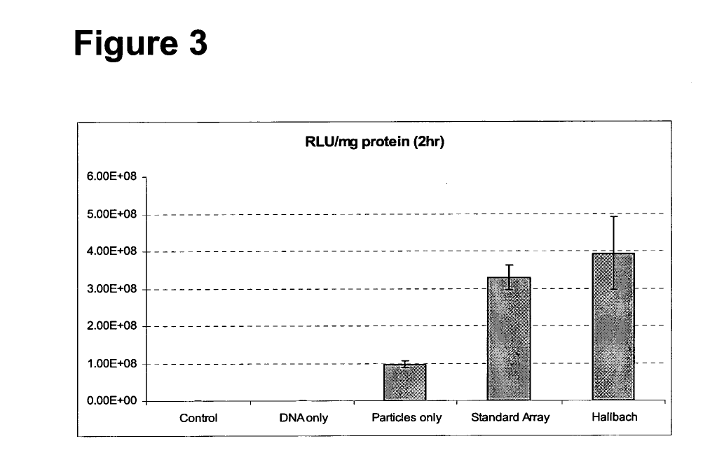

NCI-H292 (human lung epithelial) cells were maintained in RPMI 1640 culture media supplemented with 10% foetal calf serum, 100 U/mL penicillin, 0.1 mg/mL streptomycin, 0.25 μg/mL amphortericin B and 2 mM L-glutamine. Cells were seeded at 5×10³cells/well in 96 well tissue culture plates and incubated overnight at 37° C. 5% CO₂ to allow the cells to attach. Polymag® transfections (particle diameter=100-200 nm) were performed in serum-free (SF) RPMI medium using 0.1 μg DNA per well following the manufacturers recommended protocol based upon 1 μL Polymag per μg DNA. Following the addition of reagents, the plates were transferred to an incubator at 37° C. 5% CO₂ and placed above 5-magnet, oscillating (f=2 Hz, Amplitude=200 μm) NdFeB Halbach array for 2 hr. At 2 hr post transfection, the media was replaced with an equal volume of RPMI 1640 culture media supplemented with 10% foetal calf serum, 100 U/mL penicillin, 0.1 mg/mL streptomycin, 0.25 μg/mL amphortericin B and 2 mM L-glutamine. At 48 hr post transfection, the media was removed from each well and the cells lysed by the addition of 30 μL of cell reporter lysis buffer (Roche). Samples were assayed for Luciferase activity using a Luciferase assay reagent (Promega, Madison, USA)) and the total protein concentration determined using a BCA assay reagent (Pierce, Cramlington, UK). Data shown as mean±SEM; N=10. See FIG. 3 for data.

### Example 3

Using a Redcliffe Magnetics Magscan 500, the flux density of the Halbach array was scanned at a height above the array of 3 mm. This is the equivalent level of the cells within the multiwell plate which is placed above the array. A scan of the array in the x-y plane reveals regions of highest flux density (both positive and negative) and by scanning at various heights above the array, the field gradient can also be determined. (see FIG. 4a).

The force on the particles scales with both the flux density and the field gradient according to the equation:

$${F}_{m} = \frac{{V}_{m} Δχ}{{μ}_{0}} ( B \cdot ∇ ) B .$$

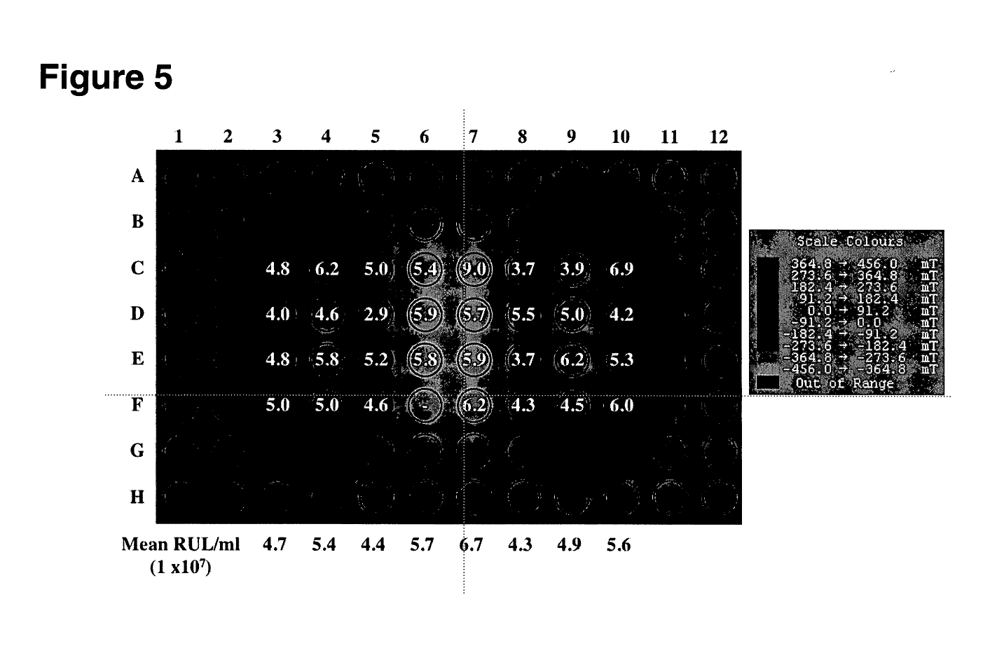

(Pankhurst et al., 2003). By using this relationship along with the field scanning data, the optimum force can be calculated for each well. In addition, this same information can be used to construct larger magnet arrays and position multiwell plates and culture flasks on the arrays so as to maximize the number of wells (or area of the flask) under optimum force conditions for a Halbach array of specific dimensions. In this case, fluorescence intensity was plotted against position on the array (see FIG. 4b and FIG. 5).

NCI-H292 (human lung epithelial) cells were maintained in RPMI 1640 culture media supplemented with 10% foetal calf serum, 100 U/mL penicillin, 0.1 mg/mL streptomycin, 0.25 μg/mL amphortericin B and 2 mM L-glutamine. Cells were seeded at 5×10³cells/well in 96 well tissue culture plates and incubated overnight at 37° C. 5% CO₂ to allow the cells to attach. Polymag® transfections (particle diameter=100-200 nm) were performed in serum-free (SF) RPMI medium using 0.1 μg DNA per well following the manufacturers recommended protocol based upon 1 μL Polymag per μg DNA. Following the addition of reagents, the plates were transferred to an incubator at 37° C. 5% CO₂ and placed above 5-magnet, oscillating (f=2 Hz, Amplitude=200 μm) NdFeB Halbach array for 2 hr. At 2 hr post transfection, the media was replaced with an equal volume of RPMI 1640 culture media supplemented with 10% foetal calf serum, 100 U/mL penicillin, 0.1 mg/mL streptomycin, 0.25 μg/mL amphortericin B and 2 mM L-glutamine. At 48 hr post transfection, the media was removed from each well and the cells lysed by the addition of 30 μL of cell reporter lysis buffer (Roche). Samples were assayed for Luciferase activity using a Luciferase assay reagent (Promega, Madison, USA)) and the total protein concentration determined using a BCA assay reagent (Pierce, Cramlington, UK).

### Example 4

The reporter genes, Green Fluorescent Protein (GFP) and luciferase, were attached to commercially available magnetic nanoparticles. Magnetic nano-particles coated with 1800 branched polyethyleneimine (PEI) were incubated with DNA in order to bind the reporter genes to the particles. The gene/particle complex was then introduced into mono-layer cultures of HEK293T kidney cells within the incubator. Culture dishes were positioned on a custom-built holder above the magnet array, housed within the incubator.

The particles were delivered using a high precision oscillating horizontal drive system controlled by a computer and custom designed control software, designed by Jon Dobson. The amplitude of the array's drive system can vary between a few nanometers to millimeters and the frequency can vary from static up to 100's of Hz.

HEK293T cells were seeded in 96 well plates at 5×10³ cells/well. The cells were transfected with 5 μg/well of 150 nm dextran/magnetite composite nanoparticles coated with PEI, loaded with pCIKLux DNA (binding capacity approx 0.2 μg DNA/μg particles). The cells were exposed to magnetic fields as shown for 24 hr post transfection, using a 5 stack of 3×NdFeB 4mm magnets per well. The cells exposed to moving field were exposed for 2 hrs at 2 Hz using a 200 μm displacement and then the magnets left in place for 22 hrs in static position.

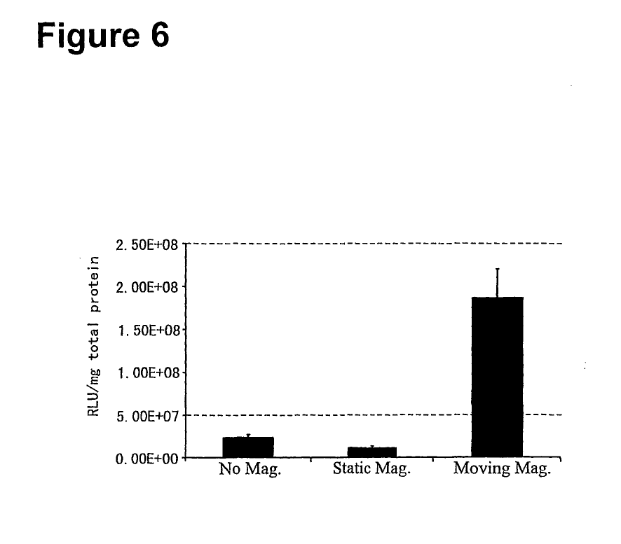

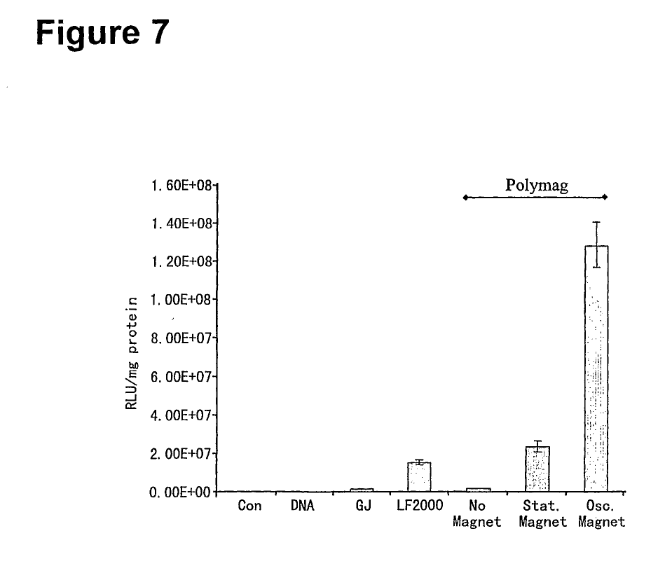

Data shown in FIGS. 6 and 7 as average +/−SEM (n=12 for each group).

The following alphabetically labeled paragraphs contain statements of broad combinations of the inventive technical features herein disclosed:

A. Device for use in magnetic transfection of cells, said device comprising magnets, and characterised in that said magnets are arranged in a Halbach array.

B. Device according to paragraph A wherein said magnets are housed in a base capable of supporting a cell-containing receptacle.

C. Device according to either paragraph A or paragraph B, wherein the base comprises an essentially flat supporting surface capable of supporting a cell-containing receptacle.

D. Device according to paragraph C, wherein the base comprises a recess capable of receiving a cell containing receptacle.

E. Device according to any preceding paragraph, wherein said base comprises a non-magnetic block comprising said magnets.

F. Device according to paragraph E, wherein the non-magnetic block is of a polymer or plastics material.

G. Device according to any preceding paragraph, wherein said magnets are NdFeB permanent magnets.

H. Device according to any preceding paragraph wherein the field strength 1 cm from the proximal surface of the Halbach array is 100 mT or greater.

I. Apparatus comprising a device according to any preceding paragraph together with a receptacle suitable for containing cells.

J. Apparatus according to paragraph I wherein the apparatus is capable of delivering a field strength of 300 mT or greater to cells during use.

K. Apparatus according to paragraph J wherein the apparatus is capable of delivering a field strength of 400 mT or greater to cells during use.

L. Device according to any of paragraphs A to H, or apparatus according to any of paragraphs I to K, wherein said receptacle is selected from the list consisting of: a multiwell plate, a cell culture flask, and a petri dish.

M. Method of transfecting cells comprising exposing said cells to magnetisable particles coupled to one or more nucleic acid or polypeptide molecules and positioning said cells within the magnetic field generated by a Halbach array.

N. Method according to paragraph M wherein the field strength to which the cells are exposed is 300 mT or greater.

O. Method according to paragraph N wherein the field strength to which the cells are exposed is 400 mT or greater.

P. Kit comprising the device of any of paragraphs 1 to H, or the apparatus of any of paragraphs I to K, together with magnetisable particles capable of being coupled to a molecule or moiety for transfection.

1. An in vitro method of delivering a reagent into a cultured cell, the method comprising positioning at least one cultured cell, and at least one magnetically susceptible particle attached to the reagent, in the magnetic field of a Halbach array such that the magnetically susceptible particle is attracted to and contacts the cultured cell.

2. The method of claim 1 further comprising the step of oscillating said magnetic field.

3. The method of claim 2 wherein the direction of oscillation is substantially perpendicular to the direction of attraction of the magnetically susceptible particles to the Halbach array.

4. The method of claim 1, wherein the cell(s) and magnetically susceptible particle(s) are positioned at one or a plurality of discrete addresses formed on a support, the method comprising aligning at least one, or at least two, of said discrete addresses with a zone of highest magnetic flux density and/or gradient of the Halbach array.

5. (canceled)

6. The method of claim 1, to 5 wherein the target cell is positioned no further than 5 mm above the surface of the array or wherein the target cell is positioned no further than 3 mm above the surface of the array.

7. (canceled)

8. The method of claims 1, wherein the reagent is chosen selected from the group consisting of: an oligonucleotide, DNA, RNA, RNAi, siRNA, an aptamer, DNA encoding a gene of interest, a nucleic acid expression construct, an amino acid, a peptide, a peptide mimetic, a protein, an antibody, an antibody fragment, an scFv, a pharmaceutical, a carbohydrate, a fatty acid and a small molecule.

9. The method of claim 1, wherein the reagent is a therapeutic agent.

10-11. (canceled)

12. The method of claim 1, wherein the cell is a bacterial, protozoan, fungal, plant or animal cell.

13. The method of claim 1 wherein the cell is mammalian.

14-15. (canceled)

16. Apparatus for the delivery of a reagent into a cell cultured in vitro, the apparatus comprising:
i) a Halbach array of magnets; and
ii) a support for positioning cells cultured in vitro in the magnetic field of the Halbach array.

17. The apparatus of claim 16 further comprising means to oscillate the magnetic field of the Halbach array.

18. The apparatus of claim 16, wherein the apparatus further comprises at least one cultured cell positioned on the surface of the support and at least one magnetically susceptible particle attached to a reagent applied to the support such that it is capable of contacting said cultured cell, wherein the magnetic field of the Halbach array is configured to attract said magnetically susceptible particle(s) towards said surface.

19. The apparatus of claim 17 wherein the apparatus further comprises at least one cultured cell positioned on the surface of the support and at least one magnetically susceptible particle attached to a reagent applied to the support such that it is capable of contacting said cultured cell, wherein the magnetic field of the Halbach array is configured to attract said magnetically susceptible particle(s) towards said surface and the means is configured to oscillate the magnetic field in a direction substantially perpendicular to the direction of attraction of the magnetically susceptible particles to the Halbach array.

20. The apparatus of claim 16, wherein one or a plurality of discrete addresses are provided on a support, the support and the Halbach array being mutually configured in the apparatus to align at least one of said discrete addresses, or at least two of said discrete addresses, with a zone of highest magnetic flux density and/or gradient of the Halbach array.

21. (canceled)

22. The apparatus of claim 20 wherein the cells and the particles are positioned at each discrete address.

23. (canceled)

24. A method of manufacturing an apparatus for the magnetic delivery of a reagent into a cell, the apparatus having a Halbach array and a support for positioning cells in the magnetic field of the Halbach array, the method comprising:
(a) providing a Halbach array;
(b) mapping the magnetic flux density and/or gradient of the magnetic field of the Halbach array;
(c) providing a cell support having one or a plurality of discrete addresses spatially configured to align with zones of highest flux density and/or gradient of the Halbach array when the support is assembled in the apparatus; and
(d) assembling the Halbach array and support to provide an apparatus for the magnetic delivery of a reagent into a cell.

25-27. (canceled)

28. A method of treatment of a human or animal in need of treatment, the method comprising delivering a reagent into a cell of an animal or human subject and having the steps of:
(i) administering a magnetically susceptible particle attached to the reagent to a tissue in the subject where treatment is required; and
(ii) positioning the tissue and magnetically susceptible particle in the magnetic field of a Halbach array such that the magnetically susceptible particle is attracted to and contacts said cells of said tissue.

29. The method of claim 28 wherein the method further comprises the step of oscillating said magnetic field.
